(19) United States
(12) Patent Application Publication
(10) Pub. No.: US 2010/0216242 A1
(43) Pub. Date: Aug. 26, 2010

(54) Cell culture support and production method and uses thereof

(75) Inventors: Kazunori Shimizu, Ritto-shi (JP); Hideaki Fujita, Nagoya-shi (JP); Eiji Nagamori, Nisshin-shi (JP)

Correspondence Address: Oliff & Berridge, PLC, Suite 500, 277 South Washington Street, Alexandria, VA, 22314, US

(73) Assignee: KABUSHIKI KAISHA TOYOTA CHUO KENKYUSHO, AICHI-GUN (JP)

(21) Appl. No.: 12/659,120

(22) Filed: Feb. 25, 2010

(30) Foreign Application Priority Data
Feb. 26, 2009 (JP) 2009-044834
Jun. 1, 2009 (JP) 2009-132301

(51) Int. Cl. C12N 5/07; H05H 1/00

(52) U.S. Cl. 435/396; 427/536

(57) ABSTRACT

The present teachings provide a practical cell culture support by which a cell culture with a high degree of freedom can be realized. More specifically, the cell culture support includes a polymer layer exhibiting thermoresponsiveness and a cell culture region obtained by plasma-treating a surface layer portion thereof with a reactive gas, whereby a cell culture support having thermoresponsiveness and cellular adhesiveness while avoiding or limiting the use of cell adhesion factors is provided.

### CROSS-REFERENCE TO RELATED APPLICATIONS

This application claims priority to Japanese Patent Application No.2009-044834 filed on Feb. 26, 2009, and to Japanese Patent Application No.2009-132301 filed on Jun. 1, 2009, the contents of which are hereby incorporated by reference into the present application.

### TECHNICAL FIELD

The present teachings provide a support with excellent cellular adhesiveness, and uses thereof.

### DESCRIPTION OF RELATED ART

It is known that poly-N-isopropylacrylamide (PNIPAAm) can form a cell culture substrate from which cells can be easily detached due to the thermoresponsiveness thereof (Japanese Examined Patent Publication No. H06-104061). For example, it has been found that a surface formed by immobilizing PNIPAAm polymer chains on a surface of a support by graft polymerization exhibits hydrophilicity at temperatures lower than the polymer phase transition temperature of 32° C. due to the development of the polymer chains, which brings their affinity for water molecules, and the surface exhibits hydrophobicity at temperatures higher than 32° C. with the polymer chains contracted. By utilizing this change in hydrophilicity toward water of the polymer chains resulting from the thermoresponsiveness thereof in such a grafted state, it becomes possible to attach and culture cells on the PNIPAAm-immobilized surface because the surface is hydrophobic at a common cell culturing temperature (37° C.), and also it becomes possible to make the surface hydrophilic at temperatures lower than 32° C. and detach the cells thereby. In other words, without performing an enzymatic treatment and the like, cultured cells can be easily detached from the surface of the culture support by utilizing the change in temperature.

On the other hand, it has been disclosed that when PNIPAAm is used as a cast product having a three-dimensional shape such as a cast film, the surface can be made adhesive by applying an aqueous solution containing PNIPAAm and a cell adhesion protein such as collagen, and drying the same to form a film (Japanese Patent Application Publication No. H03-292882).

Similarly, copolymerization of a PNIPAAm monomer and a monomer of a polymer similar to PNIPAAm with higher hydrophilicity has been carried out to impart adhesiveness to the PNIPAAm (Rochev, et al., J. Material Science: Materials in Medicine 2004, 15, 513-517). Furthermore, a copolymer of PNIPAAm monomer and gelatin has been used for the same purpose (Morikawa, et al., J Biomater. Sci. Polymer Edn. 2002, 13, 167-183).

### SUMMARY

The above prior art utilizes thermoresponsive PNIPAAm to which cellular adhesiveness has been imparted as a cell culture support. When the ease of handling of the cultured cell layer and the forming of cells into a three-dimensional shape are taken into consideration, however, such means are inadequate for utilizing thermoresponsive PNIPAAm as a cell culture support. In other words, with such means that involve the grafting of PNIPAAm to a suitable substrate surface, not only is there a problem with respect to the amount of PNIPAAm bonded to the support, but also when the grafted PNIPAAm layer reaches a predetermined thickness (e.g., tens of nanometers), it no longer exhibits cellular adhesiveness. Therefore, sophisticated control of the grafting conditions has been necessary for the growth and release of cells using a grafted PNIPAAm layer. In addition, the layers of cultured cells released from a grafted PNIPAAm layer have unstable properties, and they are extremely fragile. Furthermore, not only has it been impossible to impart a desired thickness to the grafted PNIPAAm layer, but it has also been impossible to use such a PNIPAAm layer as a sacrificial layer (i.e., a layer formed under the premise that it will be removed in a later process step) for imparting a three-dimensional shape with bridging members to a culture product.

A PNIPAAm layer having a controlled thickness and cellular adhesiveness can be formed by means using a cell adhesion factor such as a cell adhesion protein, etc. However, not only are cell adhesion factors very expensive, but there are problems with their stability because they are of biological origin, so their use has not been practical. In addition, with a copolymer of a PNIPAAm monomer and a hydrophilic monomer, it is not easy to obtain good cellular adhesiveness, the control of temperature responsiveness is difficult, and complex preprocessing (chemical synthesis) is required.

Therefore, until now a practical cell culture support that provides good cellular adhesiveness while maintaining thermoresponsiveness, and that can realize cell culturing with a high degree of freedom has not been obtained.

The present teachings provide a practical cell culture support that can attain cell cultures with a high degree of freedom and to provide uses thereof.

The inventors conducted various investigations using thermoresponsive polymers, and they first discovered that cellular adhesiveness can be attained by plasma treatment of the polymer layer with a reactive gas. In addition, they discovered that even when that cellular adhesiveness was attained, the thermoresponsiveness of the polymer layer remained unchanged. Based on these findings, the inventors completed the teachings disclosed herein. The present teachings may provide the followings.

In one aspect of the present teachings, a cell culture support comprises a polymer layer exhibiting thermoresponsiveness, and a cell culture region obtained by plasma-treating the surface layer portion of the above polymer layer with a reactive gas.

In another aspect of the present teachings, a set of cell culture supports is provided. The set comprises: (a) a first cell culture support having a polymer layer exhibiting thermoresponsiveness and a first cell culture region obtained by plasma-treating a surface layer portion of the above polymer layer with a reactive gas; and (b) a second cell culture support capable of maintaining a solid phase at a temperature at which at least the above polymer layer dissolves according to the above thermoresponsiveness thereof, the second cell culture support having a second cell culture region. In this set, the above first cell culture support and the above second cell culture support are positioned on a base material such that the above first cell culture region and the above second cell culture region are continuously arranged.

In another aspect of the present teachings, a process for producing the cell culture support may be provided. This production method comprises a step of preparing a polymer layer exhibiting thermoresponsiveness and a step of carrying out a plasma treatment with a reactive gas on at least a portion of the surface layer portion of the above polymer layer to form a cell culture region thereon.

In another aspect of the present teachings, a process for producing a set of cell culture supports may be presented. The set comprises (a) a first cell culture support having a polymer layer exhibiting thermoresponsiveness and a first cell culture region obtained by plasma-treating a surface layer portion of the above polymer layer with a reactive gas; and (b) a second cell culture support capable of maintaining a solid phase at a temperature at which at least the above polymer layer dissolves according to the above thermoresponsiveness thereof, the second cell culture support having a second cell culture region. The process comprises a step in which the above second cell culture support is prepared on a base material, then a polymer layer precursor having same composition as the above polymer layer is formed around and on a surface of the above second cell culture support, and the above first cell culture region is formed to be continuous with the above second cell culture region through abrasion of the above polymer layer precursor by the above plasma treatment, and the first cell culture support is formed.

In another aspect of the present teachings, a process for producing a cell structure may be provided. This method comprises a step of preparing one or more cultured cell units having cultured cell layers in each of which cultured cells are mutually interconnected and retained on at least a portion of the above cell culture region of the cell culture support of the present teachings, and a step of dissolving the above polymer layer by applying a temperature condition to the above polymer layer of the above cultured cell unit based on a critical solution temperature of the above polymer layer.

In another aspect of the present teachings, another process for producing a cell structure is provided. The process comprises: a step of preparing one or more cultured cell units having cultured cell layers in each of which cultured cells are mutually interconnected and retained across a continuous first cell culture region and a second cell culture region, continuously arranged with the first cell culture region, of a set of cell culture supports of the present teachings; and a step of dissolving a polymer layer of a first cell culture support of the one or more cultured cell units by applying a temperature condition to a polymer layer of the above first cell culture support based on a critical solution temperature of the polymer layer.

In another aspect of the present teachings, a cultured cell unit comprising the cell culture support of the present teachings and a cultured cell layer in which cultured cells are mutually interconnected and retained on at least a portion of a cell culture region is provided. Furthermore, in another aspect of the present teachings, a layered product obtained by superposing two or more cultured cell units, each comprising the cell culture support of the present teachings and a cultured cell layer in which cultured cells are mutually interconnected and retained on at least a portion of a cell culture region is provided. Furthermore, in another aspect of the present teachings, a cultured cell unit comprising the set of cell culture supports of the present teachings and a cultured cell layer in which cultured cells are mutually interconnected and retained across a first cell culture region and a second cell culture region that is continuously arranged with the first cell culture region.

### BRIEF DESCRIPTION OF DRAWINGS

FIG. 1 illustrates an example of a cell culture support;

FIG. 2 illustrates an example of manufacturing steps of the cell culture support;

FIG. 3 illustrates an alternative example of a utility configuration of the cell culture support;

FIG. 4 illustrates another alternative example of a utility configuration of the cell culture support;

FIG. 5 illustrates yet another alternative example of a utility configuration of the cell culture support;

FIG. 6 illustrates an example of manufacturing steps of a set of cell culture supports;

FIG. 7 illustrates an example of the manufacturing steps of a cell structure using the cell culture support;

FIG. 8 is a graph comparing contact angles of water before and after plasma treatment of a polymer layer;

FIG. 9 presents graphs of spectra showing a distribution of organic functional groups before and after plasma treatment of the polymer layer;

FIG. 10A shows a result of quantitative evaluation regarding a relationship between cellular adhesiveness and applied power, and FIG. 10B shows a result of quantitative evaluation regarding a relationship between cellular adhesiveness and duration of treatment;

FIG. 11 illustrates results of evaluation of cellular adhesiveness resulting from plasma-treating PNIPAAm layers with different molecular weights;

FIG. 12 shows a film formed by the plasma treatment;

FIG. 13 shows results of SEM observation of the film;

FIG. 14 shows results of cell patterning; and

FIG. 15 shows results of cell orientation.

### DETAILED DESCRIPTION OF THE PREFERRED EMBODIMENT

The present teachings relate to a cell culture support. The cell culture support of the present teachings may comprise a polymer layer exhibiting thermoresponsiveness and a cell culture region obtainable by plasma-treating a surface layer portion of the polymer layer with a reactive gas. The present teachings enable cellular adhesiveness to be imparted to the surface layer portion of the polymer layer, and enable the cell culture region to be formed by plasma-treating the polymer layer with the reactive gas. In addition, because the polymer layer has thermoresponsiveness, by applying temperature conditions corresponding to a phase transition temperature thereof, it is possible to make the polymer layer hydrophilic and dissolve it away in an aqueous medium, whereby the polymer layer loses its function as the cell culture region and releases the cultured cell layer. The cell culture support of the present teachings can provide a cell culture support with thermoresponsiveness and a cell culture region in which a use of molecules of biological origin such as cell adhesion factors, etc., has been avoided or limited.

By making the polymer layer into the cast product comprising a thermoresponsive polymer, the use of the base material to anchor the polymer layer can be eliminated, and the cell culture support can be provided that is capable of forming a cultured cell unit that can be handled as is without requiring the cultured cell layer obtained by culturing in the cell culture region to be physically detached therefrom.

In addition, by making the polymer layer into a cast product, it is possible not only to impart a desired shape and size to the cell culture support, but also to selectively form a cell culture region on the surface thereof by plasma treatment. As a result, the support can be used, for example as a sacrificial layer to obtain a cell structure with bridging members because the cells can be cultured using a polymer layer with a controlled thickness, and then the polymer layer can be dissolved away by utilizing its thermoresponsive properties.

In this cell culture support, the above polymer layer preferably has no cellular adhesiveness at a culturing temperature of cells. The above polymer layer is preferably a cast product comprising an acrylamide polymer. In addition, the above cell culture region may have a rough texture capable of orienting and culturing cells. The aforesaid rough texture may also be termed a concavo-convex texture. Furthermore, preferably the support comprises a conductive base material, with the above polymer layer being provided on top of the above conductive base material. Moreover, the above reactive gas preferably comprises oxygen. Additionally, the above surface layer portion of the above polymer layer may comprise a low-thermoresponsive layer including the above cell culture region and having decreased thermoresponsiveness.

A plurality of aforementioned cell culture supports may be used as a set. In this set of cell culture supports, a first cell culture region and a second cell culture region may be arranged on substantially the same plane. Preferably, the above second cell culture support has silicon as a primary component thereof.

The present teachings relate to a process for producing the aforesaid cell culture support. The process for producing the cell culture support of the present teachings enables a cell culture region to be easily imparted on the support by a plasma treatment. When the cast product is used as the polymer layer, not only can a support with excellent handling properties be fabricated, but a support having a component capable of functioning as a sacrificial layer can also be easily manufactured.

In this process, the cast product may be fabricated on a non-hydrophilic surface capable of releasing the cast product. Alternatively, a step of preparing the polymer layer may include a step of imparting a rough texture to the cast product. The rough texture is capable of orienting and culturing cells on the side contacting the above non-hydrophilic surface of the above cast product. In the process for producing the cell culture support of the present teachings, a step of forming the cell culture region may carry out the plasma treatment on the surface layer portion to an extent to form a layer having cellular adhesiveness and decreased thermoresponsiveness. In this embodiment, the process may further provide a step of dissolving the above polymer layer by applying a temperature condition thereto based on a critical solution temperature of the polymer layer.

The present teachings also relate to a process of producing the aforesaid set of cell culture supports. This production process includes a step in which the second cell culture support is prepared on a base material, then a polymer layer precursor having same composition as the above polymer layer is formed around and on a surface of the above second cell culture support, and the above first cell culture region is formed to be continuous with the above second cell culture region through abrasion of the above polymer layer precursor by the above plasma treatment, and the first cell culture support is formed. The above polymer layer precursor may preferably be plasma treated up to a point that the second cell culture region is exposed. Preferably, the treatment may be carried out so that the above first cell culture region and the above second cell culture region are arranged on substantially a same plane. In addition or alternatively, preferably the above base material is a conductive base material.

The present teachings may also be related to a process for producing the aforesaid cell structure. This method may include a step of superposing one or more of cultured cell units. This step of superposing is carried out prior to a step of dissolving the polymer layer by applying a temperature condition to the polymer layer of the cultured cell unit based on a critical solution temperature of the above polymer layer. Furthermore, in addition or alternatively, the above dissolving step may be a step of simultaneously dissolving two or more polymer layers comprised by two or more of the aforesaid cultured cell units.

The present teachings also relate to uses of the cell culture support and, more specifically, it relates to a process for producing a cultured cell complex, a cell structure, and the like. These teachings can provide a cultured cell complex with excellent handling properties. Furthermore, because a cell culture with a high degree of freedom can be realized thereby, the process for producing a cell structure enables any desired cell structure to be easily fabricated.

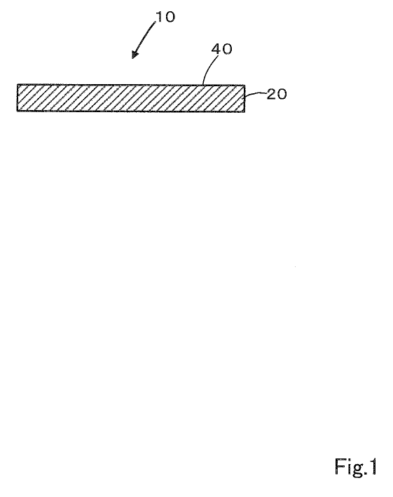

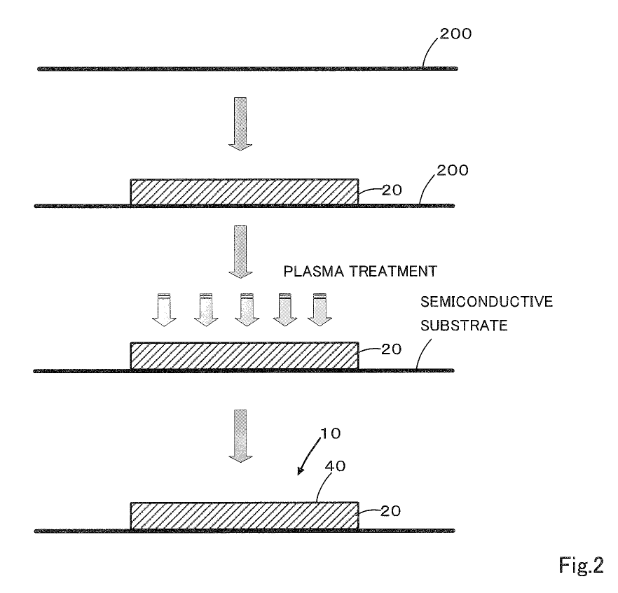

Various embodiments of the present teachings are described below with reference to the drawings as needed. These various embodiments of the present teachings are disclosed in detail. FIG. 1 illustrates an example of a cell culture support of the present teachings, and FIG. 2 illustrates an example of production steps therefor. The drawings, however, only illustrate single examples for disclosing the various embodiments, and the present teachings are by no means limited thereto.

### (Cell Culture Support)

The cell culture support of the present teachings is useful for culturing adhesive cells and configuring a structure comprising cultured cells. The cells to be cultured in the cell culture support 10 of the present teachings are not particularly limited herein so long as they are adhesive cells; however human or nonhuman animal cells are preferred. Examples of adhesive cells include: fibroblasts, myoblasts, myotube cells, corneal cells, vascular endothelial cells, smooth muscle cells, cardiomyocytes, dermal cells, epidermal cells, mucosal epithelial cells, mesenchymal stem cells, ES cells, iPS cells, osteoblasts, osteocytes, chondrocytes, fat cells, neurons, hair root cells, dental pulp stem cells, β-cells, hepatocytes, etc. In the description herein the term “cells” refers not only to individual cells, but also includes cells constituting tissues collected from the body.

When the use of these cells in regenerative medicine in humans and the like is taken into consideration, preferably, autologous cells will be used. Cells of heterozoic origin can be used as long as they provide acceptable immunocompatibility, and among allogeneic cells, either heterologous or autologous cells can be used.

### (Polymer Layer)

The cell culture support 10 of the present teachings comprises a polymer layer 20 exhibiting thermoresponsiveness. The polymer layer 20 comprises at least a thermoresponsive polymer. The thermoresponsive polymer that can be used in the present teachings exhibits hydrophobicity at cell culture temperatures (normally about 37° C.), and exhibits hydrophilicity at a temperature at which the sheet of cultured cells is collected. When the stress on the cells at the time of detachment is taken into consideration, a thermoresponsive polymer with a low critical solution temperature (T) is preferred. The term “low critical solution temperature” is defined as a phase transition temperature, below which the thermoresponsive polymer exhibits hydrophilicity and at or above which the thermoresponsive polymer exhibits hydrophobicity. In the present teachings, the lower critical solution temperature (T) is preferably between 0° C. and 80° C., more preferably, between 20° C. and 50° C., and even more preferably between 25° C. and 35° C.

The thermoresponsive polymer is not particularly limited herein, and a variety of publicly known polymers or copolymers can be used as the thermoresponsive polymer. These polymers can be crosslinked as needed, but only to an extent that properties of the thermoresponsive polymer are not lost. Examples of the thermoresponsive polymer include various polyacrylamide derivatives such as poly-N-isopropylacrylamide) (PNIPAAm), poly-N,N′-diethyl acrylamide, etc. More specifically, the following acrylamide polymers can be noted: poly-N-isopropylacrylamide (T=32° C.), poly-N-n-propylacrylamide (T=21° C.), poly-N-n-propylmethacrylamide (T=32° C.), poly-N-ethoxyethylacrylamide (T=approx. 35° C.), poly-N-tetrahydrofurfurylacrylamide (T=approx. 28° C.), poly-N-tetrahydrofurfurylmethacrylamide (T=approx. 35° C.), and poly-N,N-diethylacrylamide (T=32° C.). Examples of other polymers include poly-N-ethylacrylamide; poly-N-isopropylmethacrylamide; poly-N-cyclopropylacrylamide; poly-N-cyclopropylmethacrylamide; poly-N-acryloyl pyrrolidine; poly-N-acryloyl piperidine; polymethyl vinyl ether; alkyl-substituted cellulose derivatives such as methylcellulose, ethylcellulose, and hydroxypropylcellulose; and polyalkylene oxide block copolymers typified by a block copolymer of polypropylene oxide and polyethylene oxide. These polymers are prepared using e.g., a homopolymeric or copolymeric monomer wherein a homopolymer of the monomer has a value of T=0 to 80° C. Examples of the monomer include: (meth)acrylamide compounds; N-(or N,N-di) alkyl-substituted (meth)acrylamide derivatives; (meth)acrylamide derivatives with a cyclic group; and vinyl ether derivatives. One or more types thereof can be used. A different type of monomer other than the above can also be added and copolymerized as needed. Furthermore, a graft or block copolymer of the above polymer used in the present teachings and a different polymer, or a polymer blend of the polymer of the present teachings and a different polymer can also be used.

From the standpoint of lower critical solution temperature, etc., poly-N-isopropylacrylamide (PNIPAAm), poly-N,N′-diethylacrylamide, etc., can be preferably used as the thermoresponsive polymer.

The polymer layer 20 is preferably a layer such that cells to be cultured will not have adhesiveness thereto at the culture temperature of the aforesaid cells. The present teachings are advantageous because cellular adhesiveness can easily be imparted to such a polymer layer 20 to form a cell culture region. Such a surface property, enables e.g., seeding of the cells to be cultured so that they will reach a controlled range of cell density, and they can be cultured and observed under normal conditions. In addition, evaluation is possible by measuring the contact angle of water, etc. The polymer layer can be said to have the above surface properties when the contact angle of water is 40° or greater. The contact angle can be measured by the θ/2 method. In the case of the θ/2 method, measurement is preferably performed 1 minute after dripping 5 μL of pure water at 50° C. onto the surface of the polymer layer maintained at 50° C.

The polymer layer 20 can be a cast product of the thermoresponsive polymer. When the polymer layer 20 is casted, it can comprise a desired three-dimensional shape, and both shape and size can easily be imparted thereto as needed. By using a suitable mask, etc., a cell culture region can be selectively formed on a desired surface of the cast product. The size, shape, etc., of the thermoresponsive polymer cast product is not particularly limited herein, and the shape and size necessary for culturing the cells can be suitably determined. Typically various three-dimensional shapes such as a sheet, solid cylinder, hollow cylinder, etc., can be noted. For imparting the desired shape, a polymer solution can be dried and cured, or solution polymerization can accompany the curing associated with the polymerization of a composition containing polymerization components such as a polymerizable monomer, prepolymer, etc. Additionally, it can accompany crosslinking by heating, irradiation, etc.

When the cell culture support 10 is to be used for handling cultured cells, it is preferable for the cast product to have sufficient strength and rigidity for handling. Even more preferable, the cast product is a free-standing cast product. In this description, the term “free-standing cast product” refers to an independent cast product capable of standing on its own, or those that can be handled without any additional support. By using such a free-standing cast product, the polymer layer 20 is not used as a layer separating the layer of cultured cells from the base material, but it can support the layer of cultured cells by itself and can be used for handling purposes.

When the free-standing properties, handling properties, and layering properties of the cast product 2 itself are taken into consideration, the thickness thereof preferably lies between 1 μm and 1 mm. If the thickness is less than 1 μm, the free-standing property is difficult to maintain and the handling properties are too poor. If the thickness exceeds 1 mm, tracking to a suitable position, ease of positioning, and ease of layering are too poor. Even more preferably, the thickness lies between 10 μm and 150 μm.

Making the cast product into the shape of a sheet can be advantageous. As noted below, from the standpoint of suitability for layering, it is preferable to make the cast product into the shape of a sheet for forming a complex as a multilayered structure in which two cultured cell layers 40 are superposed via the polymer layer 20. In addition, when the cast product is formed as a pliable sheet, the cultured cell+support layered product 140 obtained using that support 10 can be suitably deformed, and the layered product 140 with a rounded or twisted three-dimensional shape can be obtained thereby.

When the cast product is used as a sacrificial layer for producing the cell structure, etc., with bridging members, preferably the thickness thereof is at least 20 nm or more, more preferably 100 nm or more, and even more preferably 1000 nm or more.

The polymer layer 20 can also be obtained by grafting the thermoresponsive polymer onto the surface of a suitable base material. For the polymer layer 20, any mode in which the thermoresponsive polymer is grafted (immobilized by covalent bonds) onto the surface of the base material can be used, regardless of the process involved in achieving such a state, as long as the thermoresponsive polymer is bonded by covalent bonds to the surface of the base material. Such grafting is carried out on the surface of the base material in the presence of a monomer, oligomer, prepolymer, or polymer by irradiation-induced polymerization wherein radiation is normally delivered as α-rays, β-rays, γ-rays, an electron beam, ultraviolet light, etc. It is known that if the thickness of such a polymer layer 20 exceeds several tens of nanometers, the cellular adhesiveness thereof is decreased. A plasma treatment restores cellular adhesiveness even in the polymer layer 20 formed by such a grafting process.

The polymer layer 20 can contain polymers and monomers other than the thermoresponsive polymer or monomer thereof as noted above. This does not eliminate the inclusion of extracellular matrix (ECM), which contains cell adhesion proteins, in the polymer layer 20 of the present teachings. It is known that sufficient cellular adhesiveness is attained in the present teachings by the plasma treatment described below, but there are cases in which an inclusion of such a component is useful for promoting growth (which includes adhesion) of the cultured cells, for structural reinforcement of the structure after release, and for maintaining cell orientation, etc. Cell adhesion proteins (peptides) in addition to the molecules present in ECM are also encompassed by the ECM component in the present description.

Such ECM components are not particularly limited herein, and various publicly known components can be used therefor. Examples include collagen, elastin, proteoglycans, glucosaminoglycans (hyaluronic acid, chondroitin sulfate, dermatan sulfate, heparan sulfate, heparin, keratin sulfate, etc.), fibronectin, laminin, hydronectin, gelatin, etc. In addition, RGD peptide, RGDS peptide, GRGD peptide, and GRGDS peptide can also be noted.

### (Cell Culture Region)

The cell culture support 10 of the present teachings comprises a cell culture region 40 on at least one part of the surface layer portion of the polymer layer 20. The cell culture region 40 is an area obtained by plasma-treating the surface layer portion of the polymer layer 20 with a reactive gas. It is believed that as a result of the plasma treatment using the reactive gas, some kind of phenomenon occurs on the surface layer portion of the polymer layer 20 that makes the treated region hydrophilic. Consequently, cellular adhesiveness sufficient to enable cell culture is exhibited thereby. The contact angle of water in the cell culture region 40 is preferably 30° or less, more preferably 20° or less, and even more preferably 10° or less.

The surface layer portion of the polymer layer 20 can comprise a low-thermoresponsive layer (in which the thermoresponsiveness is decreased) containing the cell culture region 40. This low-thermoresponsive layer is formed by the plasma treatment, and the low-thermoresponsive layer is formed with a certain thickness over an area corresponding to the plasma-treated region. The low-thermoresponsive layer has cellular adhesiveness at the plasma treated surface, and as described below, when separated from the polymer layer 20 as a film, also has cellular adhesiveness on the surface on the opposite side of the plasma treated surface.

The low-thermoresponsive layer has lower thermoresponsiveness than the polymer layer 20 lying underneath it (toward the interior). As a result, after the low-thermoresponsive layer has been formed, as long as temperature conditions corresponding to the phase transition temperature of the polymer layer 20 are not applied thereto, the low-thermoresponsive layer forms a unit with the polymer layer 20 and serves at least as one part of the surface layer portion of the polymer layer 20. On the other hand, when the temperature conditions corresponding to the phase transition temperature of the polymer layer 20 are applied, the low-thermoresponsive layer remains as a film without dissolving because the thermoresponsiveness thereof is decreased, and it can be obtained as a cell adhesive film. Because the polymer components of the polymer layer 20 and the functional groups in the polymer have been plasma-polymerized by the delivery of plasma to the surface layer portion of the plasma layer 20, it is assumed that the low-thermoresponsive layer on the plasma layer 20 assumes a composition, etc., different from that of the original polymer layer 20. At least it has been observed that the content of oxygen-containing functional groups such as O—C═O, C—O—C═O, and C—O—C increases therein. It is believed that these functional groups contribute to the increase in hydrophilicity. In addition, it is believed that C—O—C═O and C—O—C are involved in the degree of polymerization of the polymer layer 20.

The low-thermoresponsive layer is separated as a cell adhesive film by temperature conditions that cause the polymer layer 20 to dissolve. As long as it has cellular adhesiveness and can be separated as a thin film, the thickness and strength of the low-thermoresponsive layer are not particularly limited herein; and, for example, the thickness after it is isolated as a thin film (dried) is preferably 1000 nm or less, more preferably 700 nm or less, and even more preferably 500 nm or less.

Examples of the types of gases comprising the reactive gas used in the plasma treatment include oxygen (O₂) and nitrogen (N₂). Oxygen is preferred. The plasma treatment is preferably carried out on the polymer layer 20 that is deposited on a conductive base material. By so doing, cellular adhesiveness can be imparted to the surface layer portion of the polymer layer 20 with a plasma treatment of lower applied power and/or shorter duration. For example, a semiconductor base material such as Si, InAs, PbS, PbSe, etc., is preferred as the conductive base material.

The efficiency of the plasma treatment tends to decrease if the size of the polymer layer is too large when the plasma treatment is performed. It is believed that this is because the polymer layer is an insulator. Therefore, it is preferable to reduce the size of the plasma layer enough to control the decrease in plasma treatment effectiveness. When a poorly conductive or insulating substrate is used for supporting the polymer layer, it is preferable to reduce the size of such a substrate in the same manner. Preferably the surface area of the polymer layer or substrate is about 30 mm×30 mm (900 mm²) or less, and more preferably 10 mm×10 mm (100 mm²).

The applied power conditions and duration of the plasma treatment, as well as the flow rate of the reactive gas, are not particularly limited herein, and these matters can be determined as needed in accordance with the type of polymer, etc., of the polymer layer 20 to be treated, the adhesiveness, etc., of the cells to be cultured, and the like. Conditions such as increasing the applied power, decreasing the oxygen flow rate, extending the duration of treatment, decreasing the size of the substrate, and whether the substrate is conductive or not, are all believed to be involved in imparting cellular adhesiveness. In addition, a microdevice utilizing cells in combination with a separate Micro Electro Mechanical System (MEMS) component fabricated on such a substrate can be provided by preparing the cell culture support 100 on a conductive or semiconductive substrate.

The cell culture region 40 formed on the polymer layer 20 can be properly selected by suitably selecting the area for plasma treatment. The plasma treatment can be carried out by scanning, or a mask normally used in photolithography can be used to select the area for the plasma treatment.

The cell culture region 40 of the polymer layer 20 can be provided with a rough texture capable of orienting and culturing cells. The aforesaid rough texture may include, e.g., a concavo-convex texture. The cells of the cultured cell layer can be functionally oriented by providing such a rough texture. The shape thereof can be suitably established to match the orientation and alignment to be imparted to the cultured cells. In this description, the concept of “orienting cells” refers to imparting a desired directionality to cells, and it can also include imparting a desired state of alignment to the cells. Preferably the rough texture is present on essentially the entirety of the cell culture region 40, and alternatively, it can also be present on only a part thereof. It can also extend beyond the cell culture region 40.

For example, when the goal is to impart a desired directionality to the cultured cells, groove-shaped portions (which can be short in length), etc., running along the aforesaid desired direction may be provided. Additionally, when the goal is to impart a fixed state of alignment, for example, to align the entirety linearly in a desired direction, linear groove-shaped portions, etc., can be provided such that they extend across the entirety of the cell culture region 40 in the desired direction.

Such a rough texture can be formed in the cell culture region 40 concurrently with the plasma treatment, or it can be imparted to the polymer layer 20 before the plasma treatment. Such a rough texture can be imparted to the cast product by various types of etching techniques used for producing semiconductors and MEMS, or it can be imparted to a cast product by a molded body prepared using a mold fabricated by such techniques. The rough texture obtained by such means is preferred because of its high regularity. The preferred form of the concave obtained by such means is a long groove with a crosswise width between 5 μm and 100 μm. If the above width is less than 5 μm, the cells do not orient and they will not align. If it exceeds 100 μm, it is difficult for the cells to become oriented in the lengthwise axis of the concave. A more preferred width is between 5 μm and 50 μm.

As a different rough texture, an abraded surface shape obtained by machining a metal surface in a single direction can also be used. Such a rough texture itself has lower regularity than one obtained by MEMS technology, etc., but it exhibits excellent cell orientation properties. For example, a mode utilizing a surface pattern that matches the pattern of a machined metal surface as the pattern in the rough texture can be noted. In addition, a mode can be noted that utilizes a surface pattern that is the inverse of the machined metal surface as the pattern for the rough texture. Effective cell alignment tends to be easier with a rough texture having a pattern that is the inverse of the machined metal surface than with a rough texture having identical pattern to the pattern of the machined metal surface. The method for forming such a rough texture is described in detail in a paragraph below.

The surface roughness (Rz) of the rough texture forming area utilizing such a machined metal surface is preferably 2 μm or more on average. If it is less than 2 μm, orientation of the cells becomes difficult. It is preferable for the upper limit to be 20 μm or less, and more preferably 15 μm or less. The surface roughness (Rz) is a parameter defined as the mean height of ten measurement points. The reference length is suitably established to suit the surface roughness. Preferably it is obtained as the mean value of a plurality of 2 or more measurement points.

When the cultured cells are cells such as cardiomyocytes, myoblasts, myotubule cells, smooth muscle cells, and the like wherein the orientation and alignment of the cells contribute to differentiation and expression of cellular function, it is preferable to provide a cell culture region 40 comprising this rough texture.

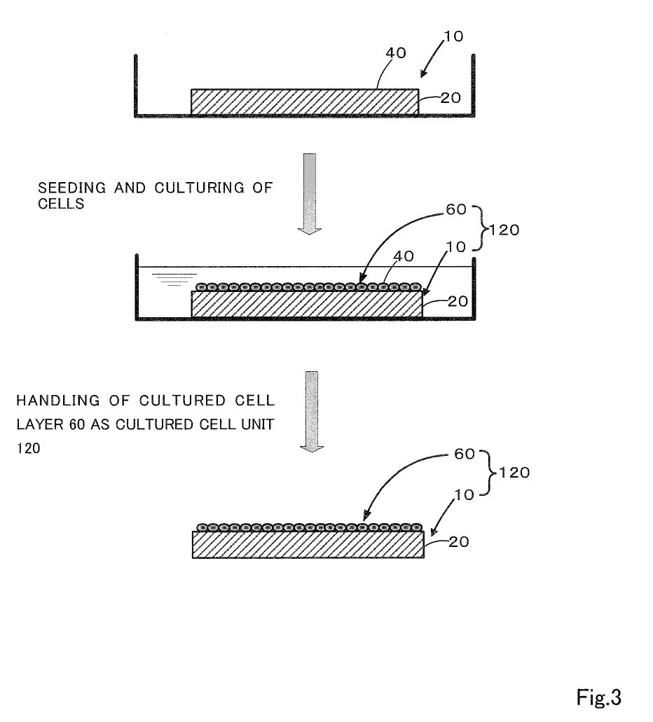

The cell culture support 10 of the present teachings disclosed above can be used as a support to culture cells. When the polymer layer 20 is a cast product, etc., and capable of being handled, as shown in FIG. 3, the cell culture support 10 enables handling of the cultured cell layer 60 as a cultured cell unit 120 comprising the cell culture support 10 and the cultured cell layer 60 wherein the cultured cells are mutually interconnected and retained in at least part of the cell culture region 40. Therefore, manipulation, transport, layering, transplantation, etc., of the cultured cell layer 60 can easily be performed without a detachment operation. Not only does this enable control or avoidance of deformation, shrinkage, and loss of orientation that are associated with the detachment and handling of the cultured cell layer 60, but it also enables the troublesome operations associated with detachment of the cell layer to be avoided.

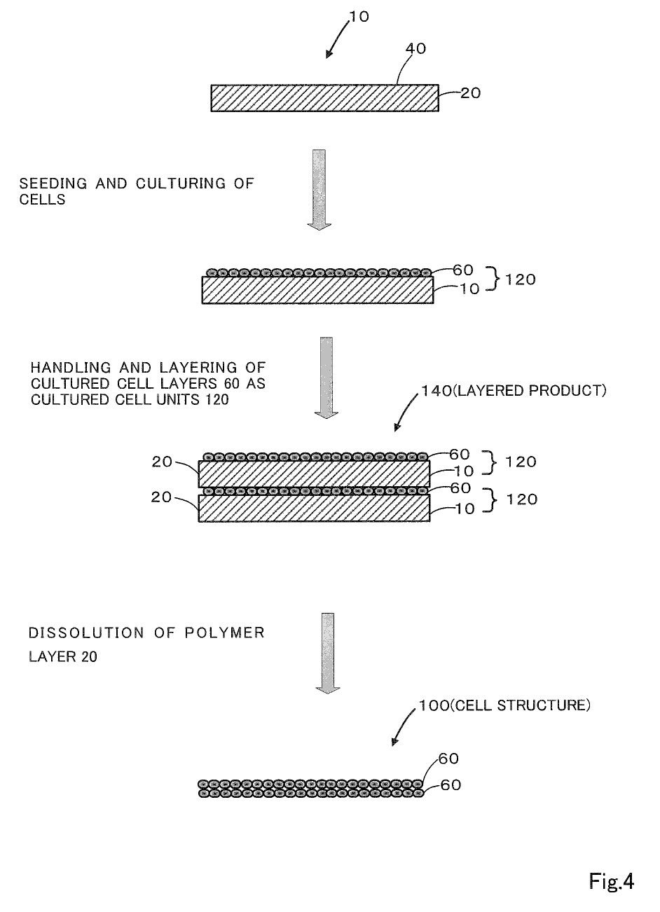

As shown in FIG. 4, the cell culture support 10 can be used for layered shaping of the cells. In other words, because the polymer layer 20 of the cell culture support 10 of the present teachings itself provides ease of handling and the cultured cell layer 60 can be handled as the cultured cell unit 120, layering with the cultured cell layers 60 becomes easy to perform. Because the polymer layers 20 can be dissolved away, a cell structure 100 comprising a laminate of cultured cell layers 60 can easily be obtained after such layering of the cultured cell units 120.

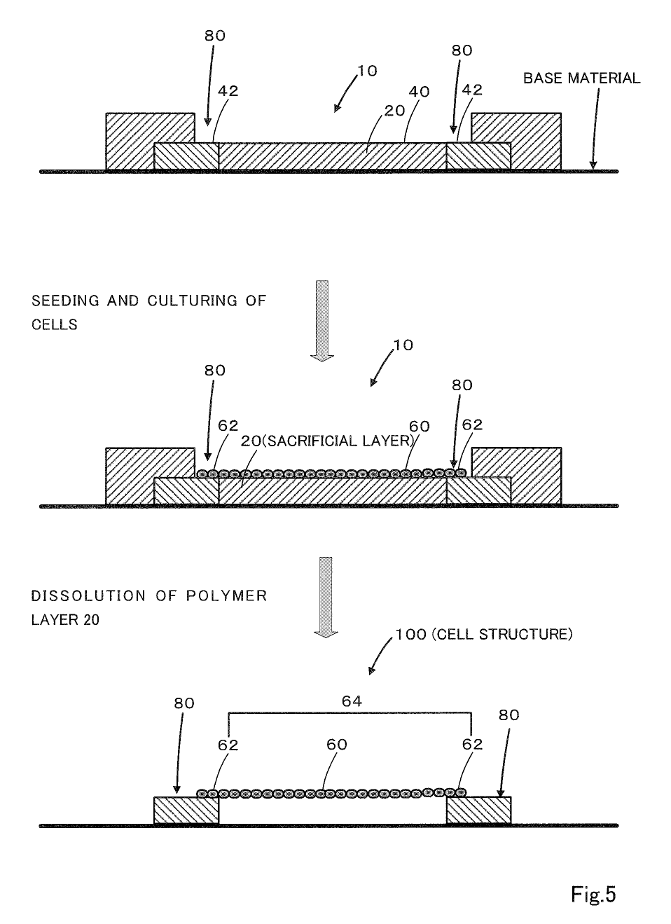

Additionally, the cell culture support 10 can be constructed so that the cell structure 100 can be fabricated comprising the cultured cell layer 60 in the shape of a bridge such as a girder (crossbeam), film, and the like wherein at least one part thereof is not supported. In other words, as shown in FIG. 5, the polymer layer 20 can be used as a sacrificial layer. More specifically, the mode of a set as will be described hereinbelow can be employed. This set includes a first cell culture support 10 and second cell culture supports 80, which comprise second cell culture regions 42 and are at least capable of maintaining solid phase at the dissolution temperature of the polymer layer 20 of the first cell culture support 10 due to the thermoresponsiveness thereof; and the above first cell culture support and the above second cell culture supports are positioned on a substrate so that the first cell culture region 40 and the second cell culture regions 42 are continuously arranged. In this mode, the cultured cell layer 60 that will become a bridging member (unsupported member) is cultured in the first cell culture region 40, and cultured cell layers 62 that will become supporting members are cultured in the second cell culture regions 42. Using such a set, a cell structure 100 comprising the cultured cell layer 60 as the bridging member and cultured cell layers 62 as the supporting members can be obtained by culturing the cells across the cell culture regions 40 and 42 to form cultured cell layers 60 and 62. The cultured cells are mutually interconnected and continuous, and the polymer layer 20 of the first cell culture support 10 is then dissolved away. In this set, if the first cell culture region 40 and the second cell culture regions 42 are arranged on substantially the same plane, a substantially-parallel bridging member can be fabricated on the substrate.

In addition, by imparting the rough texture to control orientation of the cultured cells on the surface of the first cell culture support 10, a cultured cell layer 64 with controlled orientation as the girder-shaped bridging member can be obtained. This cultured cell layer 64 is most convenient for evaluating the mechanical properties, etc., of the cultured cells or cell structure 100, and for using the cell structure 100 as a microdevice.

It is desirable for the set of cell culture supports to be provided on a conductive base material, preferably a semiconductive base material. By providing the set on the semiconductive substrate, the cell culture region 40 of the first cell culture support 10 can be formed easily, and it is most convenient for evaluating the cell structure 100 as the bridging component, and for using the same as the microdevice. The second cell culture supports 80 only need to be capable of supporting the cultured cell layers 62 and capable of at least maintaining solid phase during dissolution of the polymer layer 20 of the first cell culture support 10. However, when cellular adhesiveness and workability, etc., are taken into consideration, the use of Si, InAs, PbS, PbSe, etc., is preferred. In FIG. 5, although two second cell culture supports 80 are provided to serve as supporting members, but the present teachings are not limited thereto. A cell structure 100 having various modes of bridging members can be configured by applying means commonly used in MEMS.

The polymer layers 20 that are constituent elements of these cell culture supports and set of cell culture supports can each have a thermoresponsive layer.

By imparting phase transition temperature conditions to the polymer layer 20, the present teachings enable isolation of the low-thermoresponsive layer formed on the surface portion of the polymer layer 20 as a cell adhesive film. This cell adhesive film originates in the polymer layer 20, but it has the properties of an increased content of oxygen-containing functional groups, expression of cellular adhesiveness, and a decrease or loss of thermoresponsiveness due to the plasma treatment. Such a cell adhesive film itself can be used on the surface of the polymer layer 20 as the cell culture support with cellular adhesiveness both on the exposed surface (plasma irradiated side) and on the surface of opposite side. The cell adhesive film can be used in a variety of modes. For example, by applying it at a desired location in a cell culture vessel, etc., where cells are to be cultured, the cell culture region can easily be formed (e.g., even without a plasma irradiation apparatus, etc.). In addition, using the cellular adhesiveness thereof, the film can be layered onto the cell culture layer that has already been formed and cells can be cultured on the exposed surface thereof. Furthermore, it can be interposed between two cell culture layers to laminate and bond the same together.

### (Process for Producing Cell Culture Support)

The process for producing the cell culture support can comprise a step of preparing the polymer layer that exhibits thermoresponsiveness, and a step of forming the cell culture region by performing the plasma treatment with the reactive gas on at least one part of the surface layer portion of the above polymer layer. The process is explained below with reference as needed to FIG. 2, which is one example of the preferred manufacturing steps for producing the cell culture support 10 exemplified in FIG. 1.

### (Polymer Layer Preparation Step)

The polymer layer can be prepared as needed, but it can also be obtained commercially. The preparation step for the polymer layer 20 differs according to the configuration of the polymer layer 20. When the polymer layer 20 is formed by grafting the thermoresponsive polymer onto the suitable base material, the polymer chain is grafted onto the surface of the base material by radiation-induced polymerization in the presence of a polymer, monomer, prepolymer, etc., for grafting.

Alternatively, when the polymer layer 20 is prepared as the cast product, it is preferable to prepare a suitable polymer composition and then dry it to obtain the cast product. A polymer composition for the cast product can be prepared by dissolving the thermoresponsive polymer in a suitable solvent. Water, an organic solvent such as an alcohol, etc., that is miscible with water, and a mixed solution of water and such an organic solvent can be noted as typical examples. Normally, the preparation of the polymer composition is carried out in a temperature range at which the thermoresponsive polymer contained in the polymer composition will dissolve in the solvent used.

The forming method whereby the polymer composition is formed and made into the cast product 2 is not particularly limited herein, but in consideration of the three-dimensional shape, size, etc., of the cast product 2 to be obtained, the method can be suitably selected from among publicly known resin forming methods. For example, various publicly known methods such as the cast method, bar coat method, cap coat method, etc., can be used.

As shown in FIG. 2, to easily obtain the cast product 2, it is preferable to supply the polymer composition to a non-hydrophilic surface 200 capable of releasing the cured polymer composition, and then cure the above polymer composition. Even a thin film type of polymer layer 20 can easily be obtained in this manner. Such a non-hydrophilic surface 200 can be the forming surface of a mold having a cavity, and it can also be the surface of a flat plate as exemplified in FIG. 2. A siloxane polymer such as polydimethylsiloxane, a fluorinated polymer such as polytetrafluoroethylene, etc., can be suitably used as the material constituting the non-hydrophilic surface 200.

Various publicly known methods used for curing the thermoresponsive polymer can be used to cure the polymer composition and form the polymer layer 20 therefrom. For example, the thermoresponsive polymer can be dried under conditions wherein it dissolves in the solvent, and the solvent can be evaporated. When the cell culture support 10 is used for handling and layered shaping of the cultured cell layer 60, the polymer layer 20 should be prepared so that it has enough strength to enable handling.

Next the method for forming the rough texture for orienting and culturing cells in the cell culture region 40 of the polymer layer 20 will be described. This rough texture can be applied unchanged to the various embodiments already described, and the various methods already described can be used therefor. When forming the polymer layer 20, the rough texture is preferably imparted to the side contacting the non-hydrophilic surface 200. In other words, it is preferable to supply and cure the polymer composition on the non-hydrophilic surface that has the surface pattern for forming the rough texture pattern. Even more preferably, the rough texture is formed using the machined metal surface pattern already described. By using the machined metal surface pattern, the rough texture pattern can be easily imparted without requiring a large scale apparatus for etching technology or MEMS technology, and the rough texture can easily be imparted to a large surface area. In addition, by preparing the non-hydrophilic surface 200 having the rough texture that is identical to or the inverse of the machined metal surface pattern, a rough texture effective for cell orientation can be imparted efficiently to the polymer layer 20.

The metal used for preparing such a non-hydrophilic surface 200 is not particularly limited herein, and any metal softer than the abrasive used for machining can be used. For example, iron, aluminum, etc., can be used. In addition, the abrasive for machining can be suitably selected from publicly known abrasives, and a preferable grade size can be suitably selected from metal files and so-called sandpapers. The means of machining is not particularly limited herein, but preferably it is one enabling control of the direction of machining and the load applied at the time of machining.

The rough texture pattern can be formed to correspond to the orientation to be imparted to cells in the cultured cell layer 60 and cell structure 100 to be obtained. For orientation in essentially one direction and a linear alignment, machining should be carried out in one direction. Alternatively, machining should be carried out in accordance with the cell structure 100, etc., to be configured.

### (Formation of Cell Culture Region)

The plasma treatment with the reactive gas is carried out on the resulting plasma layer 20 to form the cell culture region 40. It is believed that cellular adhesiveness can be imparted to the polymer layer 20 by making the polymer layer 20 hydrophilic with the plasma treatment. The plasma treatment conditions can be suitably implemented in a mode already described with regard to the reactive gas and treatment conditions, etc., used in plasma treatments. For example, the conditions can be established based on an assessment of the contact angle of water on the polymer layer 20 and an assessment of the cellular adhesiveness using suitable cells. As already described, the plasma treatment can be carried out so that the contact angle of water on the plasma layer 20 is 30° or less, more preferably 20° or less, and even more preferably 10° or less. The oxygen content of functional groups determined by x-ray photoelectron spectroscopy (XPS), etc., can be used as an indicator.

The polymer layer 20 can be imparted with a suitable level of hydrophilicity and cellular adhesiveness by the plasma treatment, and thermoresponsiveness for cell release is retained at least within the range wherein cellular adhesiveness is imparted. The composition of the functional groups in the polymer chains of the thermoresponsive polymer is believed to contribute greatly to the thermoresponsiveness, and before the present application was filed, the fact that the plasma treatment can make the thermoresponsive polymer hydrophilic enough to enable cell culture without substantially inhibiting its inherent thermoresponsiveness was completely unknown. When the low-thermoresponsive layer is formed, the thermoresponsiveness is retained on the side of the layer below that layer, and it can be said that the detachability of the cultured cell layer (including the thin film originating from the low-thermoresponsive layer) is essentially assured thereby.

With the plasma treatment using the reactive gas such as oxygen, etc., the polymer layer 20 can be machined by abrasion. More specifically, shaping of the polymer layer 20 can be carried out concurrently with the plasma treatment. For example, a concave can be formed on the polymer layer 20, and the bottom thereof can be made into a cell culture region 40. By forming a cell culture region 40 within a concave member of the polymer layer 20, the cell culture region 40 can easily be delineated from its surroundings, and an appropriately-patterned cultured cell layer 60 can easily be obtained. Thus, when controlling the shape and size, or the position of the cell structure 100, it is advantageous to form the cell culture region 40 with the plasma treatment on the polymer layer 20 accompanied by machining of the polymer layer 20. Among the possible options, this is extremely advantageous when using the cell culture support 10 as the sacrificial layer to obtain the cell structure 100 as the microdevice, etc.

The plasma treatment, and particularly those using oxygen, can be carried out in a state where the polymer layer 20 is in contact with the surface of an insulating base material such as glass, but preferably it will be carried out in a state wherein the polymer layer is in contact with the surface of the conductive base material, preferably a semiconductive base material such as silicon. By so doing, cellular adhesiveness can be imparted with a treatment of lower applied power and/or shorter duration. Moreover, the plasma treatment can also be performed using the non-hydrophilic surface 200 whereon the polymer layer 20 has been formed.

The conditions for forming the low-thermoresponsive layer are not particularly limited herein and, for example, the PNIPAAm polymer layer 20 can be formed by the plasma treatment, etc., using a plasma treatment apparatus (PiPi made by Yamato Material Co., Ltd.) with an applied power output of 30 W or more, an oxygen gas flow rate between 2 mL/min and 20 mL/min, and a duration between 5 min and 30 min. The conditions for forming the low-thermoresponsive layer can be verified by applying phase transition temperature conditions to the polymer layer 20 to see whether or not the layer can be separated as a thin film after the plasma treatment.

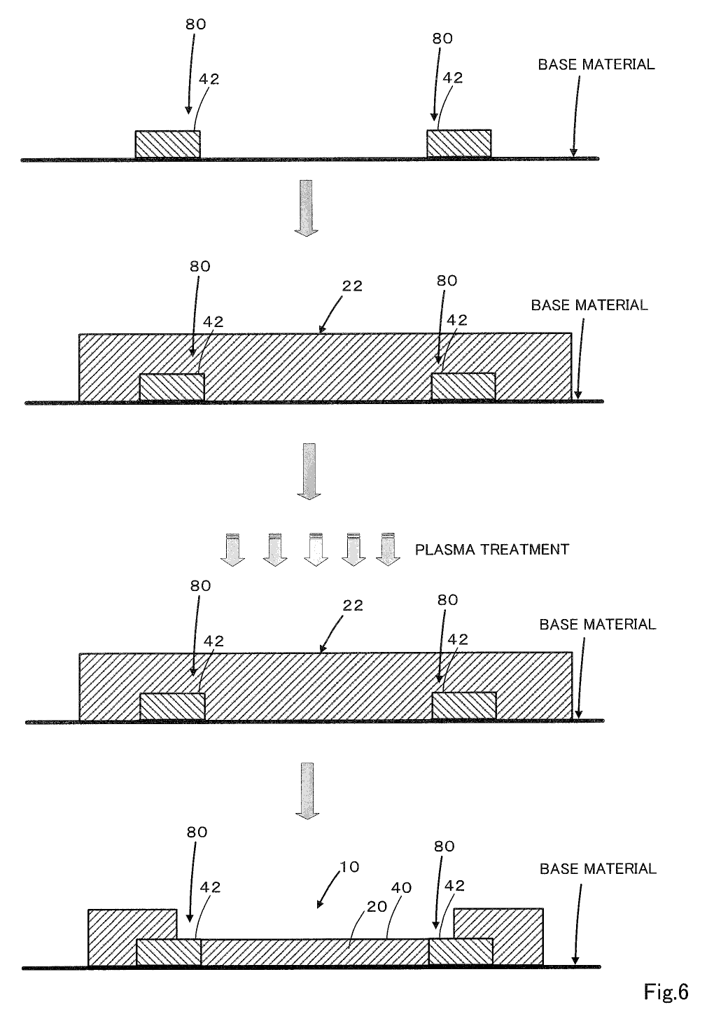

When using the cell culture support 10 to fabricate the cell structure 100 having the bridging members, there are various modes for fabricating the set of cell culture supports to obtain the bridging members. For example, one or more second cell culture support 80 is prepared on a base material, then a polymer layer precursor having the same composition as the above polymer layer is formed around and on the surface of the respective second cell culture support, and the first cell culture region 40 is formed so that it is continuously arranged with the second cell culture region 42 by abrasion of the polymer layer precursor by the plasma treatment. In other words, as shown in FIG. 6, the second cell culture supports 80, which in this case is two, but not limited thereto, are fabricated beforehand on the substrate such as silicon by MEMS technology, and then the polymer composition is supplied around and onto the respective surface of the second cell culture supports 80 on the substrate, and cured to form the precursor 22 of the polymer layer 20. Then the polymer precursor 22 can be abraded by the plasma treatment to form the first cell culture region 40 so that it is continuous with the second cell culture regions 80. The first cell culture support 10 comprising the polymer layer 20 is formed concurrently with the formation of the first cell culture region 40. Plasma treatment of the precursor 22 continues at least until the second cell culture regions 42 of the second cell culture supports 80 lying underneath the precursor are exposed. As a result, the first cell culture region 40 formed by the plasma treatment and the exposed second cell culture regions 42 easily become continuous, and both can easily be made to lie on substantially the same plane. Preferably the second cell culture supports 80 inherently comprise the second cell culture regions 42. When the second cell culture supports 80 are silicon, etc., and are abraded concurrently with the polymer layer 20, the cell culture regions 42 of the second cell culture supports 80 can function as cell culture regions 42 even if they are exposed and subsequently undergo the plasma treatment. When the plasma treatment efficiency and fabrication of the second cell culture supports 80, etc., are taken into consideration, fabrication of this set is preferably performed on the conductive based material, and more preferably on the semiconductive base material.

The process for producing the cell culture support of the present teachings described above enables the simple production using the plasma treatment of the cell culture support 10 comprising the cell culture region 40 that has both the thermoresponsiveness and cell adhesion. Therefore, an excellent cell culture support 10 can be provided inexpensively and simply without using expensive and unstable molecules of biological origin to impart cellular adhesiveness. It is also advantageous because both the shaping of the polymer layer 20 by machining and the formation of the cell culture region 40 can be achieved concurrently by using a plasma treatment.

In the process for producing the cell culture support, any polymer layer 20 that is a constituent element of the cell culture support 10 can have the thermoresponsive layer.

One embodiment of the present teachings includes a process for producing a cell adhesive film comprising a step of forming the low-thermoresponsive layer having the cell culture region 40 by using the plasma treatment on the surface layer portion of the polymer layer 20, and a step of obtaining the cell adhesive film corresponding to the low-thermoresponsive layer by applying phase transition temperature conditions to the polymer layer 20. This method enables easy production of the cell adhesive film providing cellular adhesiveness on both sides thereof.

### (Process for Producing Cell Structure)

The process for producing the cell structure of the present teachings comprises a step of preparing one or more cultured cell units 120 each having the cultured cell layer 60 in which cultured cells are mutually interconnected and retained on at least one part of the cell culture region 40 of the cell culture support 10, and a step of applying temperature conditions to the polymer layers 20 of the cultured cell units 120 based on the critical solution temperature thereof to dissolve the same. The process for producing the cell structure of the present teachings enables the cultured cell layers 60 to be obtained by utilizing the thermoresponsiveness of the polymer layers 20 of the cell culture supports 10 whereon the cultured cell layers 60 have been formed to dissolve away. In addition, it enables the cell structure 100 to be obtained by biologically connecting the cultured cell layers 60 to a grafting site or a different cultured cell layer. Because the cell culture regions 40 can easily be formed by the plasma treatment, the cell culture supports 10 simplify the steps for producing the cell structures 100 of a variety of shapes.

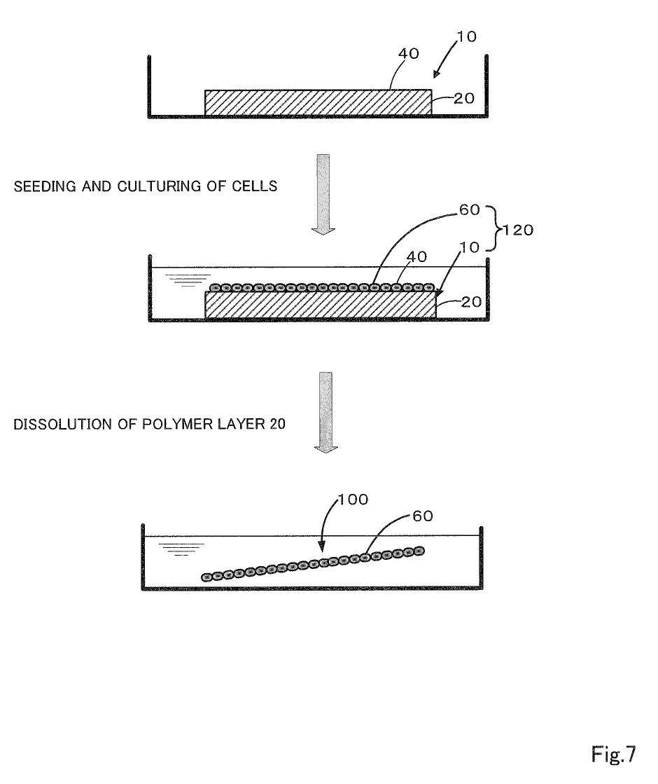

As shown in FIG. 7, the step of preparing the cultured cell unit (hereinafter, simply referred to as “unit”) involves culturing cells in the cell culture region 40 of the cell culture support 10 to form the cultured cell layer 60. Typically, the cell culture support 10 is positioned in a culturing apparatus capable of storing liquid culture medium, etc., and in the presence of the medium, cells or tissue fragments are supplied to the cell culture region 40 and cultured. Because the cell culture region 40 has cellular adhesiveness, the cells can adhere to that area 40 and grow. The culturing conditions can be suitably established by a person skilled in the art in accordance with the type of cells used, etc. Based on the lower critical solution temperature of the thermoresponsive polymer used in the polymer layer 20 of the cell culture support 10, a temperature at which the polymer layer 20 will not dissolve is established as the culturing condition. The unit 120 comprising the cell culture region 40 and the cell culture support 10 is obtained by such a culturing step.

For easily handling the unit 120 it is preferable that the polymer layer 20 have a strength greater than a predetermined level, and it is also preferable to place the cell culture support 10 on top of the surface that is non-hydrophilic in relation to the polymer layer 20 (the non-hydrophilic surface 200 used when forming the polymer layer 20) and culture the cells thereon. By so doing, the unit 120 can be easily removed from the culture system and handled.

The dissolution step is performed by applying to the polymer layer 20 temperature conditions based on the lower critical solution temperature of, for example, the thermoresponsive polymer used in the polymer layer 20 in a medium where the thermoresponsive polymer of the polymer layer 20 will dissolve or disperse. The method of applying the temperature conditions to the unit 120 is not particularly limited herein. Because the thermoresponsive polymer exhibits hydrophilicity at a temperature below the lower critical solution temperature, normally the desired temperature conditions can be applied to the polymer layer 20 in water or a solvent having water as the primary component thereof. As shown in FIG. 7, the polymer layer 20 can be broken down most simply by adjusting the temperature of the solution in which the cell culture support 10 is present. In addition, desired temperature conditions can be selectively applied to the unit 120 or the polymer layer 20 forming a part thereof by supplying a temperature-controlled gas thereto. By applying the desired temperature conditions, the polymer layer 20 dissolves in the culture medium, etc., and as a result the cultured cell layer 60 can be obtained as the cell structure 100.

When the low-thermoresponsive layer containing the cell culture region 40 is formed on the surface layer portion of the polymer layer 20, the polymer layer 20 on the side under the low-thermoresponsive layer will dissolve when the desired temperature conditions are applied, and the low-thermoresponsive layer will emerge as a thin film. When this thin film comprises a cultured cell layer 60 on an area for cell culture region 40, the film functions as a supporting layer for the cultured cell layer 60 and can be obtained as a unit together with the cultured cell layer 60. Therefore, when the cultured cell layer 60 is formed on the low-thermoresponsive layer, the cell structure 100 comprises the cultured cell layer 60 and the cell adhesive film.

The process for producing the cell structure of the present teachings basically comprises the above culturing step and dissolution step, but various modes can be adopted depending on the way the cell culture support 10 is used. Below an embodiment where the cell structure 100 configured by layering a plurality of cultured cell layers 60 is produced and an embodiment where the cell structure 100 having the bridging members is produced will both be described.

As shown in FIG. 4, to obtain the cell structure 100 by layering, a step in which one or more units are layered is performed before the dissolution step. In other words, the plurality of units 120 are layered to obtain the layered product 140 as the precursor to the cell structure 100, and then the two or more polymer layers 20 that make up the layered product 140 are dissolved. Preferably the polymer layers 20 of the two or more units 120 are dissolved simultaneously. The cells layered via the polymer layers 20 are biologically connected by dissolution of the polymer layers 20. A cell structure 100 with much better structural stability and orientation can be obtained by using the layered product 140 than by directly layering the cultured cell layers 60 themselves. Additionally, an in vitro or in vivo device, with a structure utilizing or substituting for the function of the cells or tissues, can be easily produced thereby.

The layered product 140 is obtained by layering two or more cultured cell units. The layered product 140 does not necessarily comprise cultured cell layers 60 over the entire surface of the cell culture supports 10 constituting the units 120. In other words, it can comprise cultured cell layers 60 in a desired pattern in regions, etc., defined by at least part of the surfaces of the polymer layers 20, e.g., the cell culture regions 40, etc.

The layered product 140 of the present teachings can assume a variety of forms. It can have a multilayered structure wherein two cultured cell layers 60 are layered and interposed by a single polymer layer 20. More specifically, for such a layered product 140, a multilayered structure can be noted wherein two or more units 120 are layered so that the polymer layers 20 and the cultured cell layers 60 alternate. The layered product 140 can also have a multilayered structure wherein the cultured cell layers 60 of the units 120 are layered so that they are in direct contact with each other. This multilayered form is preferred when adhesion between the cultured cell layers 60 must be carried out rapidly.

In the layered product 140 it is preferable for the cultured cells in at least part of the cultured cell layers 60 to be oriented in a predetermined direction from the standpoint of differentiation and expression of cellular function. Preferably the layered product 140 comprises cultured cells oriented in essentially the same direction in the cultured cell layers 60 of two or more continuously layered units 120.

The layered product 140 can have a multilayered structure of units 120 having cultured cell layers 60 comprising the same type of cultured cells, or it can have a multilayered structure comprising cells wherein the layered cultured cell layers 60 are different. In the layered product 140 of the present teachings, it is easy to make a complex because even if the layered cultured cell layers 60 are composed of different cell types, they form a layered structure interposed by polymer layers 20. Additionally, two or more types of cells can be cultured in the same cultured cell layer 60. The layered product 140 can have a planar shape wherein the cultured cell layers 60 in the two or more layered units 120 are identical or are different.

The layered product 140 can be comprising a deformed sheet-shaped layered product 140 as a whole. For example, the layered product 140 can be one wherein a sheet-shaped unit 120 has been rolled up and made into a hollow cylinder or other cylindrical body. In addition, the layered product 140 can be one having layered units 120 with cultured cell layers 60 in a pattern corresponding to the cross-sectional shape that results from slicing the cell structure 100 to be obtained. In such a case, because the polymer layers 20 will be broken down and removed under suitable temperature conditions, the cell structure 100 can be configured as a cell structure with a complex three-dimensional shape comprising a desired exterior and interior shape. Therefore, this layered product 140 is advantageous as the precursor for the in vitro or in vivo device utilizing or substituting for the functions of cells. Additionally, with the orientation of the cultured cell layers 60 being controlled, if the orientation in the cultured cell layers 60 that are to be layered is substantially the same, it is even more advantageous as the precursor for the in vitro or in vivo device utilizing or substituting for the functions provided by cardiomyocytes, myoblasts, myotubule cells, smooth muscle cells, etc.

When the layered product 140 has the multilayered structure comprising two or more units 120, the cell structure 100 can be obtained all at once by simultaneously removing the plurality of polymer layers 20 that make up the layered product 140. In addition, the layered product 140 can comprise units 120 containing polymer layers 20 with different lower critical solution temperatures. In this case, by applying the temperature conditions with site selectivity, the polymer layers 20 at specific sites can be broken down and the cultured cell layers 60 near those specific sites can be joined, and then at a later time, different polymer layers 20 can be broken down and different cultured cell layers 60 can be joined.

By additionally carrying out layering in the cell structure 100 obtained in this manner so that the polymer layers 20 of the layered product 140 of the present teachings are in contact, a cell structure 100 with an even more complex three-dimensional shape can be configured.

As shown in FIG. 5, to fabricate the cell structure 100 comprising bridging members, the set comprising the cell culture support 10 of the present teachings for forming the cultured cell layer 60 of the bridging member, and different cell culture supports 80 for forming cultured cell layers 62 that will serve as supporting members is prepared. Cells are provided to the cell culture regions of these cell culture supports 10, 80, and cultured cell layers 60, 62 are formed thereon to form a unit 122 that contains the different cell culture supports 80. In this culture unit 122 the cultured cell layers 60, 62 form a continuous cultured cell layer 64. By dissolving only the polymer layer 20 of the first cell culture support 10 in this unit 122, a cell structure 100 can be obtained having a cultured cell layer 64 comprising the bridging member (60) including a crossbeam and film, etc., and the supporting members (62) supported by the second cell culture supports 80. Other units 120, 122 can be layered onto the unit 122 obtained using this set to form the layered product 140 and obtain the cell structure 100 thereby.

In the fabrication of the cell structure 100, the dissolution step of the polymer layer 20 is possible both in vitro and in vivo provided there is an environment wherein the polymer layer 20 is dissolved or dispersed away, and this can be properly selected to match the use of the cell structure 100. When the stability, etc., of the cell structure 100 is taken into consideration, preferably the fabrication of the cell structure 100 will be carried out by positioning the layered product 140 at the site of use of the cell structure 100. In other words, it is preferable to place the layered product 140 at the site of use (in vivo or in vitro) of the cell structure 100, and break down the polymer layer 20 at that location. By so doing, handling of the cell structure 100 itself can be avoided, and both a structural breakdown and decrease in cell orientation of the cell structure 100 can be avoided or limited.

The present teachings can provide the unit 120 and layered product 140 that are components in the process for producing such a cell structure, and a process for producing the same.

In the process for producing the cell structure disclosed above, the polymer layers 20 that are constituent elements of the cell culture supports 10 can each have a thermoresponsive layer. In addition, when the polymer layer of the cell culture support 10 has the thermoresponsive layer, the cell adhesive film can be provided to at least one cultured cell layer 60 of the cell structure 100 obtained by the process for producing the same.

### (Cell Structure)

The cell structure 100 of the present teachings is obtained by culturing cells using the cell culture support 10 of the present teachings and removing the polymer layer 20 therefrom. The three-dimensional shape of the cell structure 100 of the present teachings is not particularly limited herein. In addition to the sheet shape, through layered shaping the construct can provide the complex three-dimensional shape having hollow members, through-hole members, and the like. The cell structure 100 of the present teachings is quite useful when such a structure is important or essential for function.

In addition, in the cell structure 100 of the present teachings, the adhesive film can be unified with the cultured cell layer 60, and these units can be layered into a plurality of layers. Furthermore, the construct can be one wherein two or more cultured cell layers 60 interposed with cell adhesive films are layered. Because the cell adhesive film has decreased thermoresponsiveness, it will not dissolve even when the original phase transition temperature conditions are applied, and the function of the cultured cells in the cell structure 100 will not be lost because of the material and structure thereof.

The cell structure 100 of the present teachings can adopt a variety of forms. In other words, it can have a structure in which the cultured cells aligned in the predetermined orientation are layered, and it can comprise the bridging members. Such a cell structure 100 is useful as a cell structure for an in vitro or in vivo device in which the cell orientation is an important factor for differentiation and expression of cellular function. More specifically, it is useful for the in vitro or in vivo device utilizing or substituting for the function of muscle tissue such as smooth muscle tissue and the like wherein cell orientation is particularly important.

The cell structure 100 of the present teachings can be used in regenerative medicine and the like to substitute for various cells, tissues, and organs in humans and nonhuman animals. In particular, a portion of cardiac muscle, where the cell orientation is very important, can be used as a regenerative material. Furthermore, the cell structure 100 of the present teachings can be used in an extracorporeal actuator and the like that utilizes the function of skeletal muscle, etc.

The present teachings are described in greater detail below through examples. However, the present teachings are by no means limited to the following examples.

### Example 1

### Cell Culture in Plasma Treated PNIPAAm Layer

In this example, the plasma treatment was carried out on the PNIPAAm layer, and the effect thereof on cell culture was ascertained.

### (1) Fabrication of PNIPAAm Film

A 5 w/v % solution of PNIPAAm (Polysciences, Inc.: poly(N-isopropylacrylamide), molecular weight: approx. 40,000 (viscosity), melting point: >200° C., glass transition temperature: 85° C.) in ethanol was cast on a glass substrate (10 mm×10 mm, in part 18 mm×18 mm (Test Nos. 5, 12, and 17 only) and dried to form a film approximately 50 μm thick and the same size as the substrate. The polymer solution was applied at 50 μL/cm². In addition, the cast volume was set to 1/10, and a film (Test No. 1) with a thickness approximately 1/10 the thickness of the others (i.e., approximately 5 μm) was fabricated.

### (2) Plasma Treatment

An oxygen plasma treatment was performed under the conditions shown in Table 1 on the approximately 50 μm thick films fabricated in (1).

### (3) Cell Culture

A controlled amount of C2C12 cells were seeded onto the plasma-treated PNIPAAm film and cultured for 24 hours at 37° C. using DMEM medium (Invitrogen, Carlsbad, Calif.) (containing 10% fetal bovine serum (FBS, ICN Biomedicals, Inc., Aurora, Ohio), 100 U/mL of potassium penicillin G, and 100 μg/mL of streptomycin sulfate (Invitrogen)), and the state of cell growth was observed. In addition, cells were seeded in the same manner onto a PNIPAAm layer (Test No. 1) that was not plasma-treated. Observations were carried out with a stereomicroscope, and after nuclear and actin staining, with a fluorescence microscope. Table 1 shows the results. The symbols in Table 1 refer to the number of cells adhering and spreading out in relation to the number of cells that adhered and spread out when they were directly seeded onto a cell adhesive substrate (control cell number). A circle represents more than 50% of the control cell number, a triangle represents less than 50% of the control cell number, and an X represents 10% or less of the control cell number.

### (4) Collection of Cultured Cells

The PNIPAAm films wherein cell growth had been observed were cooled to 25° C. while still immersed in medium to see whether or not the cells could be collected. Such cells were also re-seeded and cultured to see whether or not regrowth was possible.

TABLE 1

| | Plasma treatment conditions | | | | |
|---|---|---|---|---|---|
| | Applied | | | | |
| Test | power | O₂ flow rate | | Substrate | Cell culture |
| No. | (W) | (mL/min) | Time (min) | size (mm) | results |
| 1 | 0 | 0 | 0 | 10 × 10* | X |
| 2 | 0 | 0 | 0 | 10 × 10 | X |
| 3 | 10 | 6 | 10 | 10 × 10 | X |
| 4 | 10 | 6 | 15 | 10 × 10 | X |
| 5 | 10 | 6 | 15 | 18 × 18 | X |
| 6 | 10 | 6 | 165 | 10 × 10 | Δ |
| 7 | 20 | 6 | 10 | 10 × 10 | Δ |
| 8 | 30 | 6 | 1 | 10 × 10 | X |
| 9 | 30 | 6 | 5 | 10 × 10 | Δ |
| 10 | 30 | 6 | 10 | 10 × 10 | Δ |
| 11 | 30 | 6 | 15 | 10 × 10 | ◯ |
| 12 | 30 | 6 | 15 | 18 × 18 | Δ |
| 13 | 40 | 6 | 10 | 10 × 10 | ◯ |
| 14 | 50 | 6 | 10 | 10 × 10 | ◯ |
| 15 | 60 | 6 | 10 | 10 × 10 | ◯ |
| 16 | 60 | 6 | 15 | 10 × 10 | ◯ |
| 17 | 60 | 6 | 15 | 18 × 18 | ◯ |
| 18 | 60 | 6 | 15 | 10 × 10 | ◯ |
| 19 | 70 | 6 | 10 | 10 × 10 | ◯ |
| 20 | 80 | 6 | 10 | 10 × 10 | ◯ |
| 21 | 90 | 6 | 10 | 10 × 10 | ◯ |
| 22 | 90 | 6 | 15 | 10 × 10 | ◯ |
| 23 | 100 | 6 | 10 | 10 × 10 | ◯ |
| 24 | 100 | 6 | 15 | 10 × 10 | ◯ |
| 25 | 110 | 6 | 10 | 10 × 10 | ◯ |
| 26 | 120 | 6 | 10 | 10 × 10 | ◯ |
| 27 | 150 | 6 | 15 | 10 × 10 | ◯ |
| 28 | 200 | 6 | 15 | 10 × 10 | ◯ |
| 29 | 10 | 3 | 15 | 10 × 10 | X |
| 30 | 30 | 3 | 10 | 10 × 10 | ◯ |
| 31 | 30 | 3 | 15 | 10 × 10 | ◯ |
| 32 | 60 | 3 | 15 | 10 × 10 | ◯ |
| 33 | 10 | 9 | 15 | 10 × 10 | X |
| 34 | 30 | 9 | 10 | 10 × 10 | X |
| 35 | 30 | 9 | 15 | 10 × 10 | ◯ |
| 36 | 60 | 9 | 15 | 10 × 10 | ◯ |

*Thickness of PNIPAAm = 5 μm

As shown in Table 1, with the plasma treatment using oxygen gas at a suitable applied power (approximately between 30 W and 200 W), cells treated from several minutes to several tens of minutes adhered, spread out, and grew. It was found that the effectiveness of cell adhesion, etc., increased as the applied power and duration of the treatment increased, as the oxygen flow decreased, and as the size (surface area) of the glass substrate (polymer layer) decreased. Effective cell adhesion, etc., could not be obtained without the plasma treatment even at 1/10 the film thickness (Test No. 1). This finding corroborates the finding that the plasma treatment contributes the property of cellular adhesiveness. In addition, it was found that cells that had grown can be collected from the PNIPAAm layer by decreasing the temperature, and they can also be regrown. The inventors confirmed that the effect of cell adhesion, etc., can be imparted to a PNIPAAm layer by plasma treatment not only on the cover glass, but also on the silicon substrate and a conventional cell culture dish.

From the above results, it is clear that cellular adhesiveness, etc., can be imparted and an area capable of culturing cells can be formed by the plasma treatment on the thermoresponsive polymer layer, and that the cultured cells can be collected utilizing that thermoresponsiveness.

### Example 2

### Stability of Cellular Adhesiveness from Plasma Treatment

In this example, cells were cultured on a film fabricated under controlled conditions and stored (in a desiccator at room temperature for 16 days) and on a film fabricated under the same conditions and then used immediately after fabrication. The stability of cellular adhesiveness obtained by the plasma treatment in the two films was compared by observing the state of cell growth. The fabrication conditions for the PNIPAAm film were the same as in Example 1 wherein a 5 w/v % polymer solution in ethanol was cast onto a glass substrate (10 mm×10 mm) at 50 μL/cm² and dried. The plasma treatment conditions were set to an applied power of 60 W, oxygen flow rate of 6 mL/min, and duration of 10 min on a glass substrate, and cell culturing was carried out in the same manner as in Example 1. An elemental analysis of the surfaces of these films was also performed.

The results showed no difference in cell growth between the film after storage and the film immediately after fabrication, and likewise there was no difference in the elemental composition of the films. Based on these findings, it was determined that the effect of imparting cellular adhesiveness, etc., to a thermoresponsive polymer film by a plasma treatment is stably retained.

### Example 3

### Changes in Surface Property of PNIPAAm Layer from Plasma Treatment

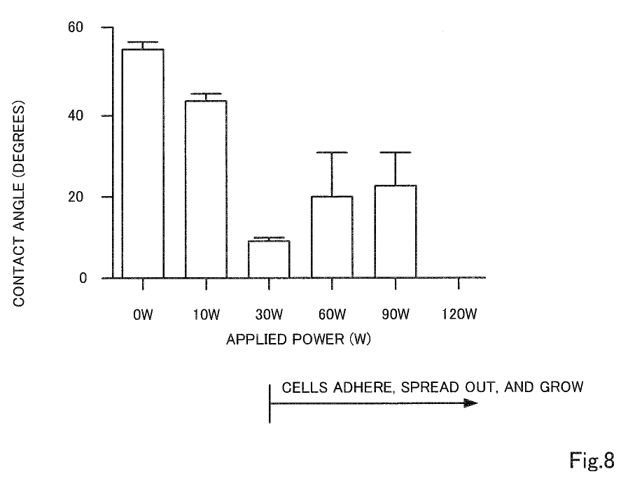

In this example the surface property (contact angle of water) of PNIPAAm films before and after plasma treatment was compared in films fabricated under controlled conditions. The cell growth was also determined in the same manner as in Example 1. The PNIPAAm film fabrication conditions were set, just as in Example 1, in which a 5 w/v % polymer solution in ethanol was cast onto a glass substrate (10 mm×10 mm) at 50 μL/cm² and dried. The plasma treatment conditions were set to an applied power of 0 W to 120 W, oxygen flow rate of 6 mL/min, and duration of 10 min on a glass substrate. The contact angle of water was measured by dripping 5 μL of pure water at 50° C. onto the PNIPAAm surface and measuring after 1 minute had elapsed. The measurement of the contact angle was performed by the θ/2 method, and the results are shown in FIG. 8.

Cell growth was excellent at an applied power of 30 W or higher. On the other hand, as shown in FIG. 8, a decrease in the contact angle was observed when the applied power was 30 W or higher. At a treatment of 60 W or higher, there was a considerable scattering of data, and the contact angle increased. Non-measurable ultra-hydrophilicity resulted from an applied power of 120 W or higher. The scattering of data and increase in the contact angle were due to the unevenness of the polymer layer formed by the plasma treatment, and it was found that cellular adhesiveness is dependent on surface hydrophilicity.

From the above it was determined that an increase in hydrophilicity of the thermoresponsive polymer layer contributes to an increase in cell adhesion. In the past hydrophilicity has been very difficult to achieve in a thermoresponsive polymer, and it was believed that hydrophilicity could not be stably maintained to the extent that cellular adhesiveness could be imparted thereto. From the above results, however, it was determined that cellular adhesiveness can be imparted by a plasma treatment even to a thermoresponsive polymer that does not inherently have cellular adhesiveness.

### Example 4

### Surface Analysis by XPS

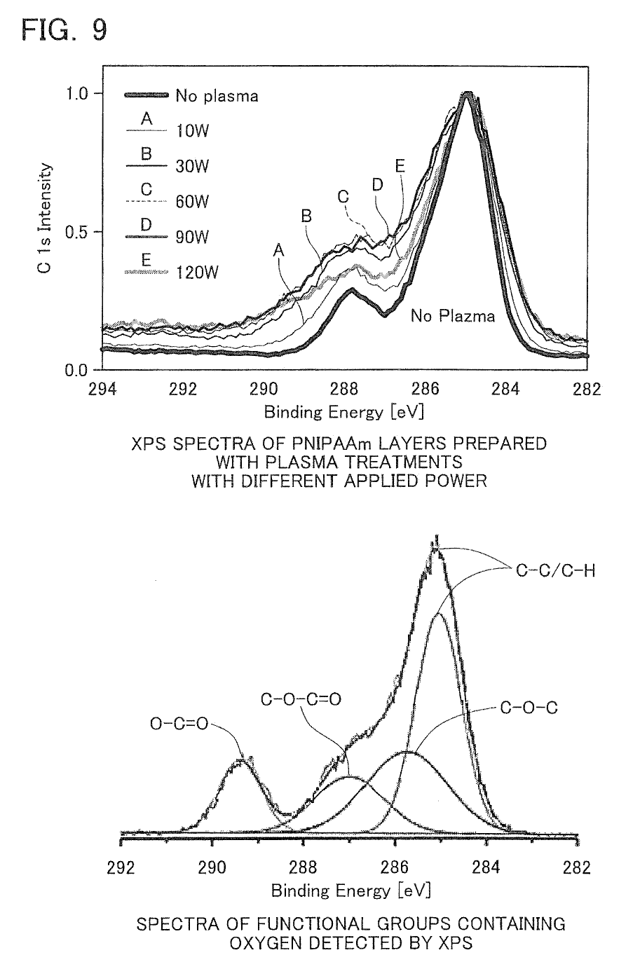

In this example the status of organic functional groups before and after the plasma treatment of Example 3 was analyzed using XPS. The results are shown in FIG. 9.

As shown in FIG. 9, peaks for C—O—C═O, C—O—C, and O—C═O were observed. The results strongly indicate polymerization of C—O—C═O and C—O—C by the plasma treatment.

### Example 5

### Quantitative Evaluation of Cellular Adhesiveness

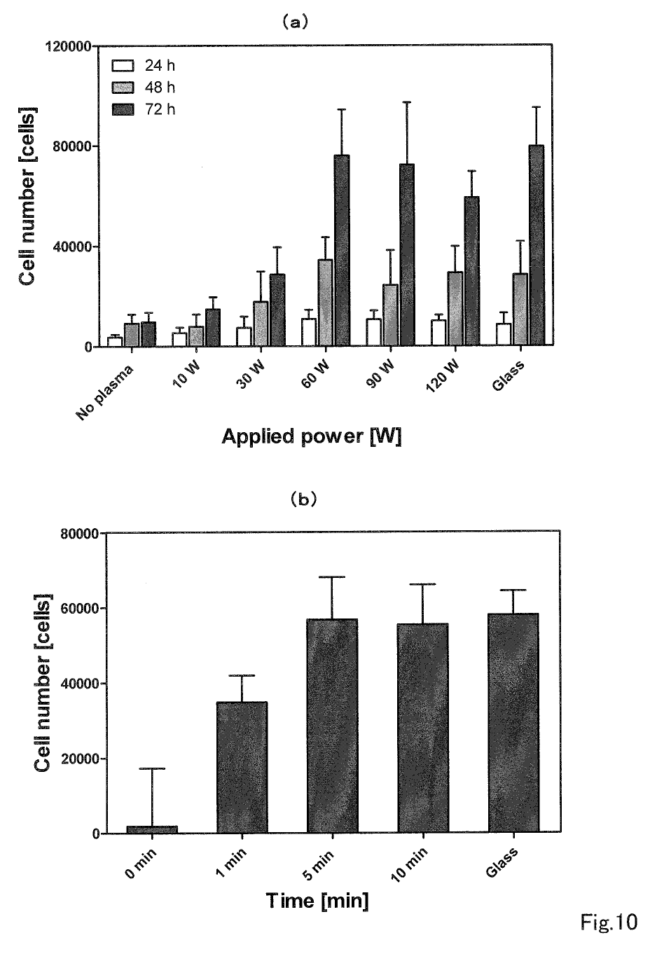

In this example the cellular adhesiveness of the surface of the PNIPAAm layer obtained by the plasma treatment was quantitatively evaluated by counting the number of cells. The PNIPAAm film fabrication conditions were set, just as in Example 1, in which a 5 w/v % polymer solution in ethanol was cast onto a glass substrate (10 mm×10 mm) at 50 μL/cm² and dried. The plasma treatment conditions for Series A were set to an applied power of 10 W to 120 W, oxygen flow rate of 6 mL/min, and duration of 10 min on a glass substrate, and for Series B were set to an applied power of 60 W, oxygen flow rate of 6 mL/min, and duration of 0 min to 10 min. A PiPi made by Yamato Material Co., Ltd., was used as the plasma treatment apparatus. For the cells, 5,000 C2C12 cells were seeded and cultured for 24 hours to 72 hours in DMEM medium in an incubator at 38° C. and 5% carbon dioxide (humidity 99%). FIG. 10 shows the results.

As shown in FIG. 10A, significant cellular adhesiveness was found with a plasma treatment at an applied power of 30 W or more, an oxygen flow rate of 6 mL/min, and a duration of 10 minutes. No trend toward a larger increase in cellular adhesiveness was seen at 60 W or more. As shown in FIG. 10B, significant cell adhesion was found with a plasma treatment of 60 W, oxygen flow rate of 6 mL/min, and duration of 1 minute or longer.

As a reference, when a plasma treatment was performed on the same PNIPAAm layer using a different plasma treatment apparatus (model PDC210, made by Yamato Material Co., Ltd.), at applied powers ranging from 150 W to 350 W (50 W increments), an oxygen flow rate of 50 mL/min, and a duration of 10 minutes, some cellular adhesiveness was finally observed at 350 W. Therefore, it was found that the plasma treatment conditions such as applied power, etc., differ greatly depending on the plasma treatment apparatus used, and the applied power, oxygen flow rate, duration of treatment, etc., must be suitably set depending on the device used.

### Example 6

### Evaluation of Cellular Adhesiveness by Plasma Treatment for PNIPAAm Layers of Different Molecular Weights

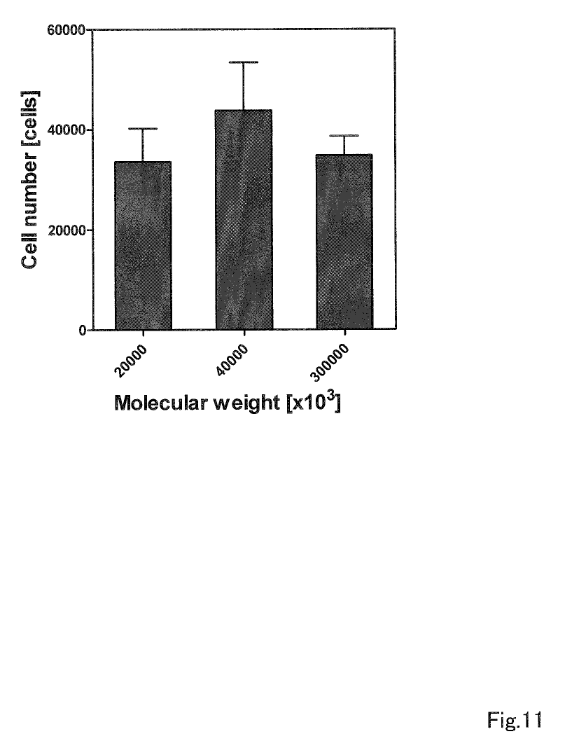

In this example PNIPAAm layers were formed in the same manner as in Example 1 using PNIPAAm of different molecular weights to see whether or not the cellular adhesiveness would be different depending on the plasma treatment. For the PNIPAAm, in addition to that used in Example 1 (made by Polysciences, Inc., molecular weight: approx. 40×10⁶), a polymer made by Aldrich with a molecular weight of approximately 20×10⁶, and one made by Scientific Polymer Products with a molecular weight of approximately 300×10⁶ were used. PNIPAAm layers were formed in the same manner as in Example 1 using these PNIPAAm products. The plasma treatment conditions were set to an applied power of 120 W, oxygen flow rate of 6 mL/min, and duration of 60 minutes. In addition, 5,000 C2C12 cells were seeded and cultured for 48 hours in DMEM medium in an incubator at 38° C. and 5% carbon dioxide (humidity 99%), and the number of cells was counted. FIG. 11 shows the results.

As shown in FIG. 11, it was determined that there is no relationship between the molecular weight of the PNIPAAm and cellular adhesiveness.

### Example 7

### Formation of Cellular Adhesiveness by Oxygen Plasma Treatment on PNIPAAm Layer

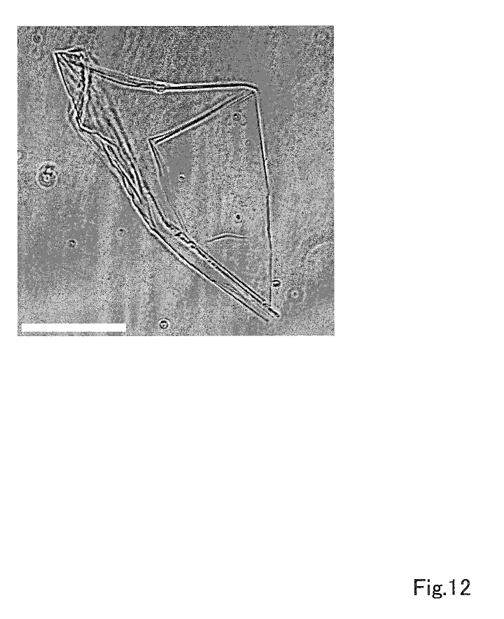

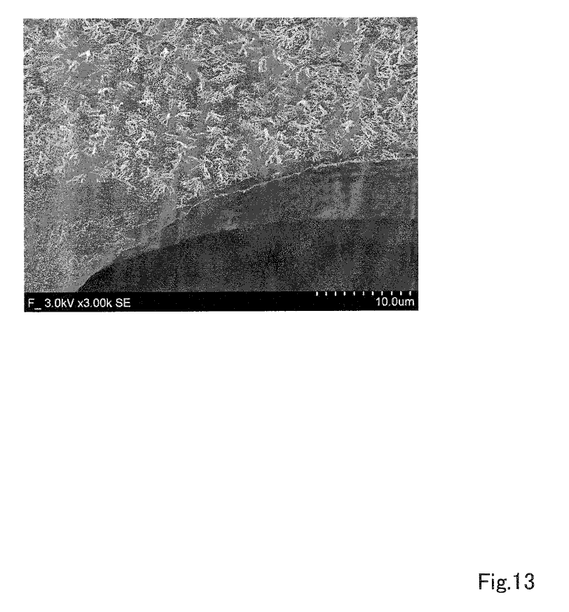

In this example it was confirmed that a layer with decreased thermoresponsiveness is formed on the PNIPAAm layer by the oxygen plasma treatment. In other words, a PNIPAAm layer was formed in the same manner as in Example 1, and a plasma treatment was performed at an applied power of 120 W, oxygen flow rate of 6 mL/min, and a duration of 5 minutes. After the resulting PNIPAAm layer (film) was immersed in water below the phase transition temperature (32° C.) of the PNIPAAm used, the body of the film was observed after the PNIPAAm layer had dissolved (see FIG. 12). FIG. 13 shows the results when the film was collected on a copper mesh and dried, and then observed by SEM. The results of the SEM observation revealed that the film is a thin film not exceeding 500 nm in thickness. Because this thin film has low thermoresponsiveness, it does not dissolve under conditions wherein the normal phase transition temperature is applied, but it does dissolve when exposed to the phase transition temperature or lower for an extremely long period of time.

### Example 8

### Imparting Patterning and Cell Orientation to Cell Culture Region

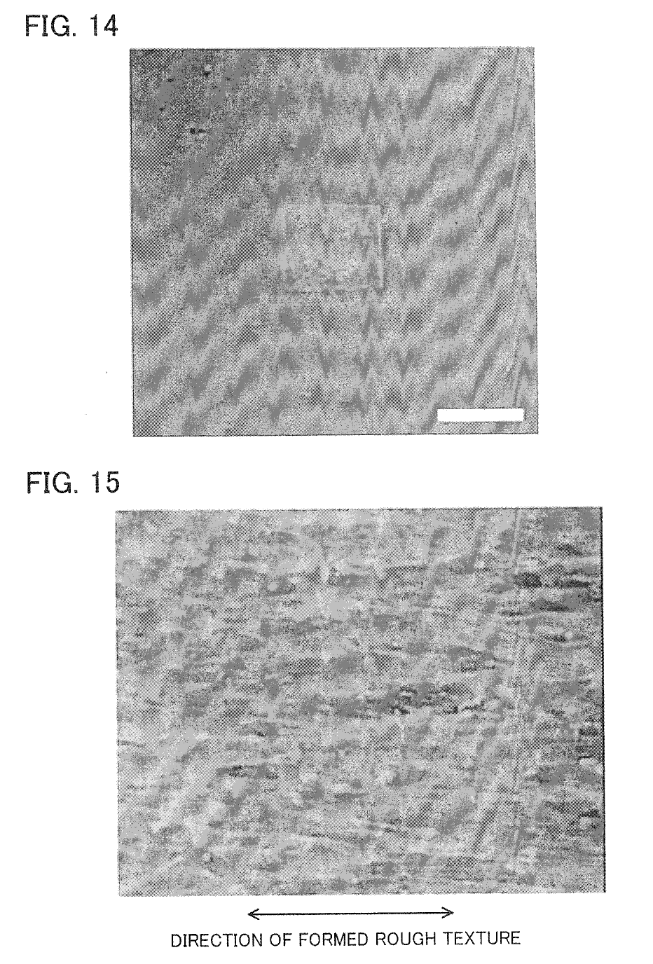

In this example a cell patterning effect and the effect of imparting a rough texture by the plasma treatment on the PNIPAAm layer were confirmed. In other words, a PNIPAAm layer was formed in the same manner as in Example 1, and using masking, a plasma treatment was performed on the center part alone at an applied power of 120 W, oxygen flow rate of 6 mL/min, and duration of 5 min. In addition, 5,000 C2C12 cells were seeded onto this PNIPAAm layer and cultured for 48 hours in DMEM medium in an incubator at 38° C. and 5% carbon dioxide (humidity 99%). The viable cells were stained with cell tracker blue and observed under a fluorescence microscope. As shown in FIG. 14, the results reveal that cells grew only in the plasma-treated center part, and cells neither adhered nor grew in the untreated parts. From this it was determined that cell patterning is possible by patterning the plasma-treated area.

Following Example 1 a PNIPAAm layer was formed by transferring a rough pattern that had been prepared on polydimethylsiloxane (PDMS) using a metal file. Then a plasma treatment was performed at an applied power of 120 W, oxygen flow rate of 6 mL/min, and duration of 5 min. In addition, 5,000 C2C12 cells were seeded onto the plasma-treated surface of this PNIPAAm layer and cultured in DMEM medium in an incubator at 38° C. and 5% carbon dioxide (humidity 99%). After the cells had grown, they were subcultured for 5 days, then immunostained for actin and observed under a fluorescence microscope. As shown in FIG. 15, from the results it was determined that orientation of the cells is possible by performing a plasma treatment on a PNIPAAm layer with a rough texture.

1. A cell culture support comprising:
a polymer layer exhibiting thermoresponsiveness; and
a cell culture region obtained by plasma-treating a surface layer portion of the polymer layer with a reactive gas.

2. The support according to claim 1, wherein the polymer layer is a layer that has no cellular adhesiveness at a culturing temperature of cells.

3. The support according to claim 1, wherein the polymer layer is a cast product comprising an acrylamide polymer.

4. The support according to of claim 1, wherein the cell culture region has a rough texture capable of orienting and culturing cells.

5. The support according to claim 1, further comprising a conductive base material, the polymer layer being provided on top of that conductive base material.

6. The support according to claim 1, wherein the surface layer portion of the polymer layer comprises a low-thermoresponsive layer containing the cell culture region and having decreased thermoresponsiveness.

7. A set of cell culture supports, the set comprising:
(a) a first cell culture support having a polymer layer exhibiting thermoresponsiveness and a first cell culture region obtained by plasma-treating a surface layer portion of the polymer layer with a reactive gas; and
(b) a second cell culture support capable of maintaining a solid phase at a temperature at which at least the polymer layer dissolves according to the thermoresponsiveness thereof, the second cell culture support having a second cell culture region,
wherein the first cell culture support and the second cell culture support are positioned on a base material such that the first cell culture region and the second cell culture region are continuously arranged.

8. The set according to claim 7, wherein the second cell culture support has silicon as a primary component thereof.

9. A process for producing a cell culture support, comprising steps of:
preparing a polymer layer exhibiting thermoresponsiveness; and
carrying out a plasma treatment with a reactive gas on at least a part of a surface layer portion of the polymer layer to form a cell culture region thereon.

10. The production process according to claim 9, wherein the polymer layer preparation step includes a step in which a cast product is fabricated on a non-hydrophilic surface capable of releasing the cast product.

11. The production process according to claim 10, wherein the polymer layer preparation step comprises imparting a rough texture to the cast product, the rough texture configured capable of orienting and culturing cells on a side contacting the non-hydrophilic surface of the cast product.

12. The production process according to claim 10, wherein the step of forming the cell culture region carries out the plasma treatment on the surface layer portion to an extent to form a layer having cellular adhesiveness and decreased thermoresponsiveness.

13. The production process according to claim 12, further comprising a step of dissolving the polymer layer by applying a temperature condition to the polymer layer based on a critical solution temperature thereof.

14. A process for producing a set of cell culture supports, the set comprising (a) a first cell culture support having a polymer layer exhibiting thermoresponsiveness and a first cell culture region obtained by plasma-treating a surface layer portion of the polymer layer with a reactive gas; and (b) a second cell culture support capable of maintaining a solid phase at a temperature at which at least the polymer layer dissolves according to the thermoresponsiveness thereof, the second cell culture support having a second cell culture region, the process comprising:
a step in which the second cell culture support is prepared on a base material, then a polymer layer precursor having the same composition as the polymer layer is formed around and on a surface of the second cell culture support, and the first cell culture region is formed to be continuous with the second cell culture region by abrasion of the polymer layer precursor by the plasma treatment, and the first cell culture support is formed.

15. A process for producing a cell structure, comprising steps of:
preparing one or more cultured cell units having cultured cell layers in which cultured cells are mutually interconnected and retained on at least a part of a cell culture region of a cell culture support including a polymer layer exhibiting thermoresponsiveness and the cell culture region obtained by plasma-treating a surface layer portion of the polymer layer with a reactive gas; and
dissolving the polymer layer of the cultured cell unit by applying a temperature condition to the polymer layer based on a critical solution temperature of the polymer layer.

16. The production process according to claim 15, further comprising a step of superposing one or more of the cultured cell units, wherein the step of superposing is carried out prior to the dissolving step.

17. A process for producing a cell structure, the process comprising steps of:
preparing one or more cultured cell units having cultured cell layers in which cultured cells are mutually interconnected and retained across a first cell culture region and a second cell culture region, arranged continuously with the first cell culture region, of a set of cell culture supports including: (a) a first cell culture support having a polymer layer exhibiting thermoresponsiveness and a first cell culture region obtained by plasma-treating a surface layer portion of the polymer layer with a reactive gas and (b) a second cell culture support capable of maintaining a solid phase at a temperature at which at least the polymer layer dissolves according to the thermoresponsiveness thereof, the second cell culture support having a second cell culture region, wherein the first cell culture support and the second cell culture support are positioned on a base material such that the first cell culture region and the second cell culture region are continuously arranged; and
dissolving the polymer layer of the first cell culture support of the cultured cell unit by applying temperature conditions to the polymer layer of the first cell culture support based on a critical solution temperature of the polymer layer of the first cell culture support.

18. A cultured cell unit, comprising:
a cell culture support including a polymer layer exhibiting thermoresponsiveness and a cell culture region obtained by plasma-treating a surface layer portion of the polymer layer with a reactive gas; and
a cultured cell layer in which cultured cells are mutually interconnected and retained on at least a part of the cell culture region.

19. A layered product obtainable by superposing two or more cultured cell units, each of which comprises a cell culture support including a polymer layer exhibiting thermoresponsiveness and a cell culture region obtained by plasma-treating a surface layer portion of the polymer layer with a reactive gas; and a cultured cell layer in which cultured cells are mutually interconnected and retained on at least a part of the cell culture region.

20. A cultured cell unit comprising a set of cell culture supports including: (a) a first cell culture support having a polymer layer exhibiting thermoresponsiveness and a first cell culture region obtained by plasma-treating a surface layer portion of the polymer layer with a reactive gas and (b) a second cell culture support capable of maintaining a solid phase at a temperature at which at least the polymer layer dissolves according to the thermoresponsiveness thereof, the second cell culture support having a second cell culture region, wherein the first cell culture support and the second cell culture support are positioned on a base material such that the first cell culture region and the second cell culture region are continuously arranged; and a cultured cell layer in which cultured cells are mutually interconnected and retained across the first cell culture region and the second cell culture region arranged continuously with the first cell culture region.
